(19) United States
(12) Patent Application Publication
(10) Pub. No.: US 2015/0126865 A1
(43) Pub. Date: May 7, 2015

(54) ULTRASONIC PROBE AND ULTRASONIC MEASURING DEVICE

(71) Applicant: SEIKO EPSON CORPORATION, Tokyo (JP)

(72) Inventors: Kiyoaki MURAI, Matsumoto-shi (JP); Michihiro NAGAISHI, Suwa-shi (JP); Akira MARUYAMA, Azumino-shi (JP)

(21) Appl. No.: 14/523,063

(22) Filed: Oct. 24, 2014

(30) Foreign Application Priority Data
Nov. 5, 2013 (JP) 2013-229058

(51) Int. Cl. A61B 5/00; A61B 8/08

(52) U.S. Cl. CPC A61B 5/0035; A61B 8/0858; A61B 5/0064
USPC 600/437

(57) ABSTRACT

An ultrasonic probe includes an ultrasonic element section for ultrasonic measurement, a first light emitting section that emits red light so as to overlap an ultrasonic measurement range, a second light emitting section that emits near-infrared light so as to overlap the ultrasonic measurement range, and a light receiving section that receives reflected light of the red light and the near-infrared light from a subcutaneous portion of the body. Measurement target tissue in the ultrasonic measurement range is detected from the strength of the received reflected light, and a notification section notifies that the measurement target tissue has been detected.

### BACKGROUND

1. Technical Field

The present invention relates to an ultrasonic probe including an ultrasonic element for ultrasonic measurement of tissue in the body.

2. Related Art

A technique for measuring biological information in the body in a non-invasive manner using an ultrasonic measuring device is known.

For example, one of the techniques is to measure the intima media thickness (IMT) of the carotid artery, which is an indicator of arteriosclerosis.

In the measurement of the carotid artery, it is necessary to find the carotid artery and determine the measurement point appropriately. Normally, the operator places an ultrasonic probe at the approximate position of the carotid artery to be measured based on medical knowledge, finds the carotid artery to be measured in detail while observing the B-mode image displayed on the monitor, and manually sets the found carotid artery as the measurement point. Skill is required for the operation to quickly find the appropriate position or posture at which the ultrasonic probe is placed. In recent years, a function to assist such a preparatory operation has come to be devised. For example, JP-A-2002-11008 discloses a method of detecting a blood vessel automatically by using the received signal strength of the reflected wave of the ultrasonic beam.

In addition, as a technique for acquiring biological information in a non-invasive manner, a technique for measuring the oxygen saturation of arterial blood is known. For example, JP-A-6-98881 and JP-A-2005-95581 disclose a technique for calculating the pulsating component ratio of the absorbance by arterial blood flow by irradiating body tissue with light beams of different wavelengths and measuring the reflected light or the transmitted light and then calculating the oxygen saturation of arterial blood from the ratio of absorbance.

In the detection method disclosed in JP-A-2002-11008, an ultrasonic array of a two-dimensional array type that includes an ultrasonic transducer column for blood vessel position detection and an ultrasonic transducer column for blood flow measurement is required. Therefore, there is a problem in that the ultrasonic probe becomes expensive.

### SUMMARY

An advantage of some aspects of the invention is to realize an auxiliary function of detecting the position of the ultrasonic measurement target at a lower cost.

A first aspect of the invention is directed to an ultrasonic probe including: an ultrasonic element section that performs ultrasonic measurement of measurement target tissue in a body; and a light emitting section and a light receiving section that emit and receive measurement light for detecting the measurement target tissue by optical measurement and that are provided such that a propagation range of the measurement light in the body overlaps a measurement range of the ultrasonic element section.

According to the first aspect of the invention, tissue as an ultrasonic measurement target (measurement target tissue) can be detected by emitting measurement light propagating through the body and receiving and measuring the reflected light from the tissue in the body. Therefore, since it is not necessary to prepare a relatively expensive ultrasonic array of a two-dimensional array type unlike in the related art, the ultrasonic probe can be made to have an auxiliary function of detecting a measurement target at a lower cost.

A second aspect of the invention is directed to the ultrasonic probe according to the first aspect of the invention, wherein the ultrasonic element section includes an ultrasonic element array, and the ultrasonic element array is disposed between the light emitting section and the light receiving section.

According to the second aspect of the invention, a line connecting the light emitting section and the light receiving section crosses the ultrasonic element array. Therefore, it is possible to detect the presence of the measurement target tissue in the appropriate positional relationship with respect to the longitudinal direction of the measurement target tissue.

In particular, when the measurement target tissue is a blood vessel, if the light emitting section and the light receiving section are disposed along the blood vessel direction, the measurement light reaches the light receiving section after long propagation of the measurement light through the blood vessel. Therefore, it is possible to detect the presence of the measurement target tissue with higher accuracy. In ultrasonic measurement for finding a blood vessel, for example, a cross section of the blood vessel (cross section perpendicular to the traveling direction of the blood vessel) is measured. This is convenient for detection of the presence of measurement target tissue and ultrasonic measurement subsequent thereto.

A third aspect of the invention is directed to the ultrasonic probe according to the first or second aspect of the invention, which further includes a notification section that notifies that the measurement target tissue has been detected by the optical measurement.

In the related art, in particular, in the technique disclosed in JP-A-2002-11008, the operator should observe an ultrasonic image displayed on the monitoring screen, which is different from using the hand to manipulate the ultrasonic probe, and read and interpret the presence of measurement target tissue from the ultrasonic image. For this reason, skill and concentration have been required in order to adjust the position of the ultrasonic probe.

However, according to the third aspect of the invention, when measurement target tissue is detected, a notification is given. Therefore, the operator has only to focus on the hand to manipulate the ultrasonic probe and does not need to read and interpret the ultrasonic image, and concentration therefor is not required. As a result, it is possible to significantly reduce the operation load of the operator.

A fourth aspect of the invention is directed to the ultrasonic probe according to the third aspect of the invention, wherein the notification section is a structural section through which light from the light emitting section leaks or is guided to a side of the ultrasonic probe, and the notification is given by controlling an emission pattern of the light emitting section.

According to the fourth aspect of the invention, since it is not necessary to provide a dedicated light emitting section or the like separately as a notification section, it is possible to further reduce the manufacturing cost.

A fifth aspect of the invention is directed to the ultrasonic probe according to any one of the first to fourth aspects of the invention, wherein the measurement target tissue is a blood vessel.

Since the fifth aspect of the invention has all the features of the first to fourth aspects of the invention, the fifth aspect of the invention is very effective for ultrasonic measurement of the blood vessel.

A sixth aspect of the invention is directed to an ultrasonic measuring device including: the ultrasonic probe according to any one of the first to fifth aspects of the invention; and a detection control section that detects the measurement target tissue by controlling the light emitting section and the light receiving section to perform the optical measurement.

According to the sixth aspect of the invention, the same effects as in any one of the first to fifth aspects of the invention are obtained.

A seventh aspect of the invention is directed to an ultrasonic measuring device including: the ultrasonic probe according to the fourth aspect of the invention; a detection control section that detects the measurement target tissue by controlling the light emitting section and the light receiving section to perform the optical measurement; and a notification control section that makes the light emitting section emit light in a predetermined emission pattern when the measurement target tissue is detected by the detection control section.

According to the seventh aspect of the invention, the same effects as in the fourth aspect of the invention are obtained.

An eighth aspect of the invention is directed to an ultrasonic measuring device including: the ultrasonic probe according to the third aspect of the invention in which the notification section is a display section; a detection control section that detects the measurement target tissue by controlling the light emitting section and the light receiving section to perform the optical measurement and calculating an index value indicating a level to detect the measurement target tissue; and a notification control section that performs display control of the notification section according to the index value calculated by the detection control section.

According to the eighth aspect of the invention, the operator can be notified of the state of the index value, which is used in determining the measurement target tissue, using the display from the notification section. Therefore, the operator can find the location of the measurement target tissue quickly and efficiently based on the display.

### BRIEF DESCRIPTION OF THE DRAWINGS

The invention will be described with reference to the accompanying drawings, wherein like numbers reference like elements.

FIG. 1 is a diagram showing an example of the system configuration of an ultrasonic measuring device.

FIGS. 2A to 2C are diagrams of three sides showing an example of the configuration of an ultrasonic probe in a first embodiment.

FIG. 3 is a diagram for explaining the principle of detecting measurement target tissue.

FIGS. 4A to 4C are diagrams for explaining the flow of the ultrasonic measurement.

FIG. 5 is a block diagram showing an example of the functional configuration of the ultrasonic measuring device of the first embodiment.

FIG. 6 is a flowchart for explaining the flow of the process according to the detection of the presence of measurement target tissue and the ultrasonic measurement in the first embodiment.

FIGS. 7A to 7C are diagrams of three sides showing an example of the configuration of an ultrasonic probe in a second embodiment.

FIG. 8 is a block diagram showing an example of the functional configuration of the ultrasonic measuring device of the second embodiment.

FIG. 9 is a flowchart for explaining the flow of the process according to the detection of the presence of measurement target tissue and the ultrasonic measurement in the second embodiment.

FIG. 10 is a flowchart continued from FIG. 9.

FIGS. 11A and 11B are diagrams showing a modification example of the configuration of the ultrasonic probe.

FIGS. 12A and 12B are diagrams showing a modification example of the configuration of the ultrasonic probe.

### DESCRIPTION OF EXEMPLARY EMBODIMENTS

### First Embodiment

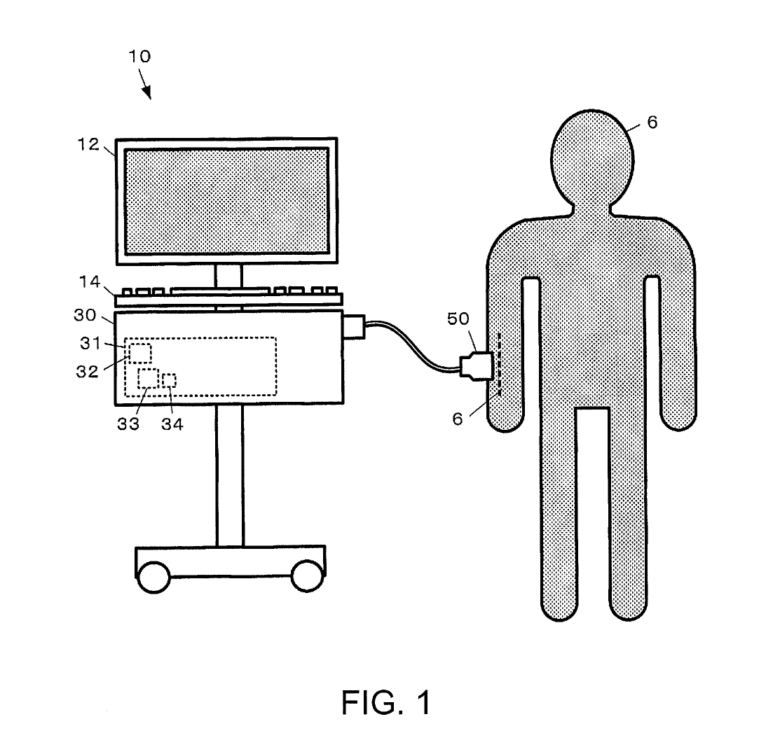

FIG. 1 is a diagram showing an example of the system configuration of an ultrasonic measuring device 10 according to the present embodiment. The ultrasonic measuring device is a device for acquiring biological information by performing ultrasonic measurement of predetermined measurement target tissue inside a body 4.

In the present embodiment, the measurement target tissue is a blood vessel, more specifically, an artery. However, other tissues may be measured. In addition, biological information to be measured can be appropriately set. Examples of the biological information to be measured include vessel diameter, an arteriosclerosis index value, an elasticity index value, blood pressure, blood vessel age, and intima media thickness (IMT).

The ultrasonic measuring device 10 includes a touch panel 12 serving as an operation input unit and a display unit for image display of a measurement result, operation information, or the like, a keyboard 14 for inputting an operation, an ultrasonic probe 50 (depth probe), and a processing unit 30. A control board 31 is mounted in the processing unit 30, so that a signal can be transmitted and received to and from each unit of the touch panel 12, the keyboard 14, the ultrasonic probe 50, and the like.

Not only a central processing unit (CPU) 32, an application specific integrated circuit (ASIC), and various kinds of large scale integration (LSI) but also a storage medium 33, such as an IC memory or a hard disk, and a communication IC 34 for realizing data communication with an external device are mounted on the control board 31. The processing unit 30 realizes the various functions according to the present embodiment by causing the CPU 32 or the like to execute a measurement program stored in the storage medium 33.

Specifically, under the control of the processing unit 30, the ultrasonic measuring device 10 transmits an ultrasonic pulse from the ultrasonic probe 50 to the body 4 and receives the reflected wave. Then, by performing amplification and signal processing of the received reflected wave, positional information, temporal change, or the like of the internal structure of the body, such as a blood vessel 6 of the body 4, is measured. As a result, biological information of interest can be sequentially calculated and stored. The reflected wave signal includes an image of each mode, such as so-called A mode, B mode, M mode, and color Doppler. Undoubtedly, the reflected wave signal may be data of other types. Measurement (sampling) using an ultrasonic wave is repeatedly performed at predetermined periods. A measurement unit is referred to as a “frame”. In the present embodiment, sampling is performed at 20 frames per second (fps) or more.

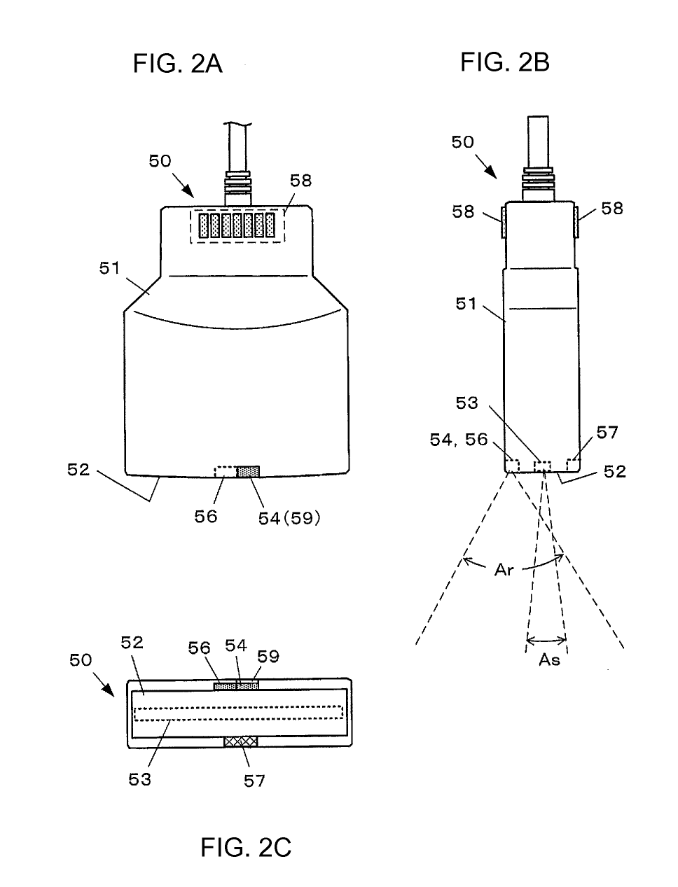

FIGS. 2A to 2C are diagrams of three sides showing an example of the configuration of the ultrasonic probe 50 in the present embodiment. FIG. 2A is a front view, FIG. 2B is a side view, and FIG. 2C is a bottom view, that is, a view seen from the side which is pressed against the skin surface of the body 4.

The ultrasonic probe 50 of the present embodiment is realized in basically the same manner as a known ultrasonic probe, but has different characteristics in terms of the following points.

That is, an ultrasonic element section 53, a first light emitting section 54, a second light emitting section 56, and a light receiving section 57 are provided on a measurement surface 52 side, to which an ultrasonic wave is emitted, so as to be formed integrally with a main body case 51. In addition, a notification section 58 is provided in an upper portion of the main body case 51. The ultrasonic measuring device 10 of the present embodiment can detect the presence of tissue, which is an ultrasonic measurement target, by receiving and measuring reflected light from the body 4 of measurement light, which is emitted from the first and second light emitting sections 54 and 56, using the light receiving section 57 and notify the operator that the presence of measurement target tissue has been detected using the notification section 58.

The ultrasonic element section 53 can be realized by an element group in which a plurality of ultrasonic transducers are arrayed in rows, for example, by a known linear array in which ultrasonic transducers are disposed in a row. In addition, the arrangement of ultrasonic transducers is not limited to one row, and the number of rows can be appropriately set according to the purpose of ultrasonic measurement.

The first and second light emitting sections 54 and 56 emit measurement light downward from the bottom surface of the main body case 51. More specifically, the first and second light emitting sections 54 and 56 are disposed such that a propagation range Ar of the measurement light overlaps a measurement range As of the ultrasonic wave by the ultrasonic element section 53.

The first light emitting section 54 is a light emitting element that emits light around 660 nm and red visible light, and emits one of two types of measurement light for detecting the measurement target tissue of ultrasonic measurement by optical measurement. For example, the first light emitting section 54 is realized by a red light emitting diode (LED), but may be realized by other light emitting elements. The first light emitting section 54 of the present embodiment is provided so as to emit measurement light in a transmission direction of the ultrasonic wave by the ultrasonic element section 53, that is, toward the normal direction of the measurement surface 52.

The second light emitting section 56 is a light emitting element that emits near-infrared light near 880 nm, and emits the other one of the two types of measurement light. For example, the second light emitting section 56 is realized by an infrared LED, but may be realized by other light emitting elements. The second light emitting section 56 of the present embodiment is also provided so as to emit measurement light in a transmission direction of the ultrasonic wave by the ultrasonic element section 53, that is, toward the normal direction of the measurement surface 52.

If one of the first and second light emitting sections 54 and 56 meets the light emission characteristics required for the other one, the other light emitting section may be omitted, and the first and second light emitting sections 54 and 56 may be realized by one light emitting element.

The light receiving section 57 is an element that receives reflected light of the probe light emitted from the first and second light emitting sections 54 and 56 and outputs a signal according to the received light strength. For example, the light receiving section 57 can be realized by an optical sensor, such as a photodiode. In the present embodiment, the light receiving section 57 is provided so that light from the normal direction of the measurement surface 52 can be received.

In the present embodiment, the first and second light emitting sections 54 and 56 and the light receiving section 57 are disposed with an ultrasonic element array interposed therebetween. Specifically, the first and second light emitting sections 54 and 56 are provided adjacent to the outer edge of the measurement surface 52 corresponding to one side of the right and left sides (top and bottom sides in FIG. 2C) of the element row of the ultrasonic element section 53. On the other hand, the light receiving section 57 is provided adjacent to the outer edge of the measurement surface 52 corresponding to the other side of the right and left sides (top and bottom sides in FIG. 2C) of the element row of the ultrasonic element section 53.

In addition, a structural section 59 is provided on the outer edge of the measurement surface 52 so that light emitted from the first light emitting section 54 leaks to the side of the main body case 51. The structural section 59 can be realized by a notch, a through hole, or a window provided in the main body case 51. Alternatively, the structural section 59 may be realized by providing a light guiding member for guiding light from the first light emitting section 54. In addition, the structural section 59 may have a configuration in which the first light emitting section 54 is exposed on the side surface.

The notification section 58 is a display unit for notifying the operator of a state (measurement status) according to the measurement of the ultrasonic measuring device 10 using light, and is realized by a light emitting element, such as a small flat panel display or LED. In the present embodiment, the notification section 58 is formed by an array of a plurality of LEDs.

The notification section 58 performs one or a plurality of kinds of notification according to a notification pattern (in the present embodiment, an emission pattern based on a combination of a blinking pattern, color, light and dark, and the like). Notification in the present embodiment includes 1) “under tissue detection determination” displayed by replacing the size of the index value indicating the likelihood of the presence of measurement target tissue (level to detect the measurement target tissue), that is, a degree of certainty with the number of LEDs that emit light, 2) “tissue detection” notifying that the measurement target tissue has been detected, and 3) “under ultrasonic measurement” notifying that ultrasonic measurement is being performed.

In addition to these three states, other states can also be appropriately included in the notification. Conversely, “under tissue detection determination” or “under ultrasonic measurement” can be omitted. In addition, although the notification using light is used in the present embodiment, notification using sound may also be used. In this case, the notification section 58 may appropriately include a speaker.

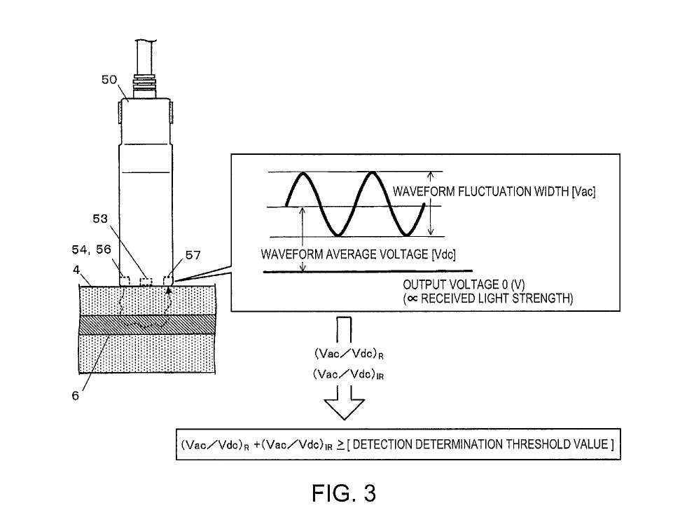

FIG. 3 is a diagram for explaining the principle of detecting measurement target tissue by the first and second light emitting sections 54 and 56 and the light receiving section 57.

When measurement light is emitted from the first and second light emitting sections 54 and 56 toward a subcutaneous portion in a state where the measurement surface 52 of the ultrasonic probe 50 is in light contact with the skin of the body 4, reflected light from the tissue below the measurement surface 52 can be measured by the light receiving section 57.

As is apparent from the technology of the known reflection type pulse oximeter, for the red light emitted from the first light emitting section 54 and the near-infrared light emitted from the second light emitting section 56, the absorbance of oxyhemoglobin in the blood and the absorbance of deoxyhemoglobin are different. In addition, the absorbance of the blood vessel 6 changes with the beating of the heart.

Focusing on the blood vessel 6 and the surrounding tissue, the fluctuation rate of reflected light (fluctuation rate of the received light strength) by the blood vessel 6 is larger than the fluctuation rate of reflected light due to the surrounding tissue of the blood vessel 6.

That is, based on the fluctuation rate of reflected light, it is possible to determine whether the reflected light received by the light receiving section 57 is due to the blood vessel 6 or the surrounding tissue, in other words, whether the blood vessel 6 is present below the skin of a position, which is in contact with the ultrasonic probe 50 at that point in time, or subcutaneous tissue is present below the skin of the position.

Specifically, it is possible to calculate a red reflected light fluctuation rate (Vac/Vdc)R, which is obtained by dividing the waveform fluctuation width (Vac) of red light by a waveform average voltage (Vdc), and near-infrared reflected light fluctuation rate (Vac/Vdc) IR, which is obtained by dividing the waveform fluctuation width (Vac) of near-infrared light by the waveform average voltage (Vdc). Then, a “detection determination parameter value { (Vac/Vdc)R+(Vac/Vdc)IR}” is calculated as a value indicating the fluctuation rate, and the detection determination parameter value is compared with a predetermined detection determination threshold value. Then, it is determined that reflected light of the blood vessel 6 is being received if the detection determination parameter value is equal to or greater than the threshold value, and it is determined that reflected light from the surrounding tissue of the blood vessel 6 is being received if the detection determination parameter value is less than the threshold value.

Incidentally, the red reflected light fluctuation rate (Vac/Vdc)R and the near-infrared reflected light fluctuation rate (Vac/Vdc) IR are also calculated in the known reflection type pulse oximeter. However, when calculating the artery oxygen saturation, a value ((Vac/Vdc) R÷ (Vac/Vdc) IR) obtained by dividing the red reflected light fluctuation rate (Vac/Vdc)R by the near-infrared reflected light fluctuation rate (Vac/Vdc) IR is used.

In contrast, the present embodiment is largely different from the known reflection type pulse oximeter in that the sum of two kinds of fluctuation rates of the red reflected light fluctuation rate and the near-infrared reflected light fluctuation rate is used for determination of blood vessel detection. By using the sum of two kinds of fluctuation rates of the red reflected light fluctuation rate and the near-infrared reflected light fluctuation rate, it is possible to improve the determination accuracy significantly compared with a case where the known reflection type pulse oximeter is used for blood vessel detection as it is.

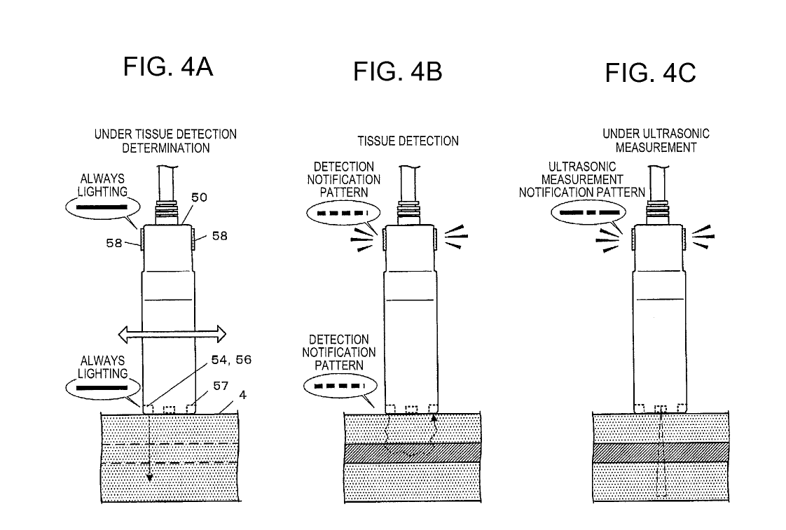

FIGS. 4A to 4C are diagrams for explaining the flow of the ultrasonic measurement in the present embodiment.

In response to a predetermined preparatory operation of the operator, as shown in FIG. 4A, the ultrasonic measuring device 10 starts the emission of measurement light, which is used in detecting the measurement target tissue, from the first and second light emitting sections 54 and 56, and further starts detection determination based on the light receiving result of the light receiving section 57. In addition, the notification section 58 level-displays the size of the detection determination parameter value used for the detection determination (index value indicating the likelihood of the presence of measurement target tissue).

The operator understands that the detection is ready by the red light emitted from the first light emitting section 54 through the structural section 59 or the notification pattern indicating “under tissue detection determination” of the notification section 58, and presses the ultrasonic probe 50 against the body 4. In addition, a display prompting the ultrasonic probe 50 to be placed at an approximate position estimated that there is measurement target tissue may be given on the touch panel 12 at the start of emission of the first and second light emitting sections 54 and 56.

The operator adjusts the contact position of the ultrasonic probe 50 so as to find the measurement target tissue (blood vessel 6). The level display of the notification section 58 depends on position adjustment.

As shown in FIG. 4B, when the ultrasonic probe 50 reaches above the blood vessel 6 eventually, the ultrasonic measuring device 10 detects this as described in FIG. 3 and sets the measurement status as “tissue detection”. The ultrasonic measuring device 10 controls the first and second light emitting sections 54 and 56 and the notification section 58 with a predetermined detection notification pattern for notification of detection (for example, an emission pattern blinking at 1 Hz). The notification section 58 may be controlled to change to a predetermined color if the emission color can be controlled.

Based on the emission pattern change of the first light emitting section 54 or the second light emitting section 56 and the notification section 58, the operator understands that the position adjustment has been completed, a blood vessel has been detected, and the ultrasonic probe 50 has been placed at an appropriate position for ultrasonic measurement. Accordingly, the operator tries to maintain the ultrasonic probe 50 at the position and posture.

When measurement target tissue is detected, as shown in FIG. 4C, the measurement status is changed to “under ultrasonic measurement”. The ultrasonic measuring device 10 makes the notification section 58 emit light in an ultrasonic measurement notification pattern for notification of the start of ultrasonic measurement (for example, an emission pattern repeating long lighting and short lighting), and automatically starts ultrasonic measurement using the ultrasonic element section 53. In addition, a display indicating that the ultrasonic measurement will start may be appropriately given on the touch panel 12 before starting the ultrasonic measurement.

Ultrasonic measurement is continued until a predetermined end condition is satisfied. The end condition can be set as “when predetermined measurement time has passed” or “measurement end operation of the operator”, for example. When it is detected that the end condition is satisfied, the ultrasonic measuring device 10 ends the ultrasonic measurement and performs control to turn off the notification section 58.

### Explanation of the Functional Configuration

Next, a functional configuration for realizing the present embodiment will be described.

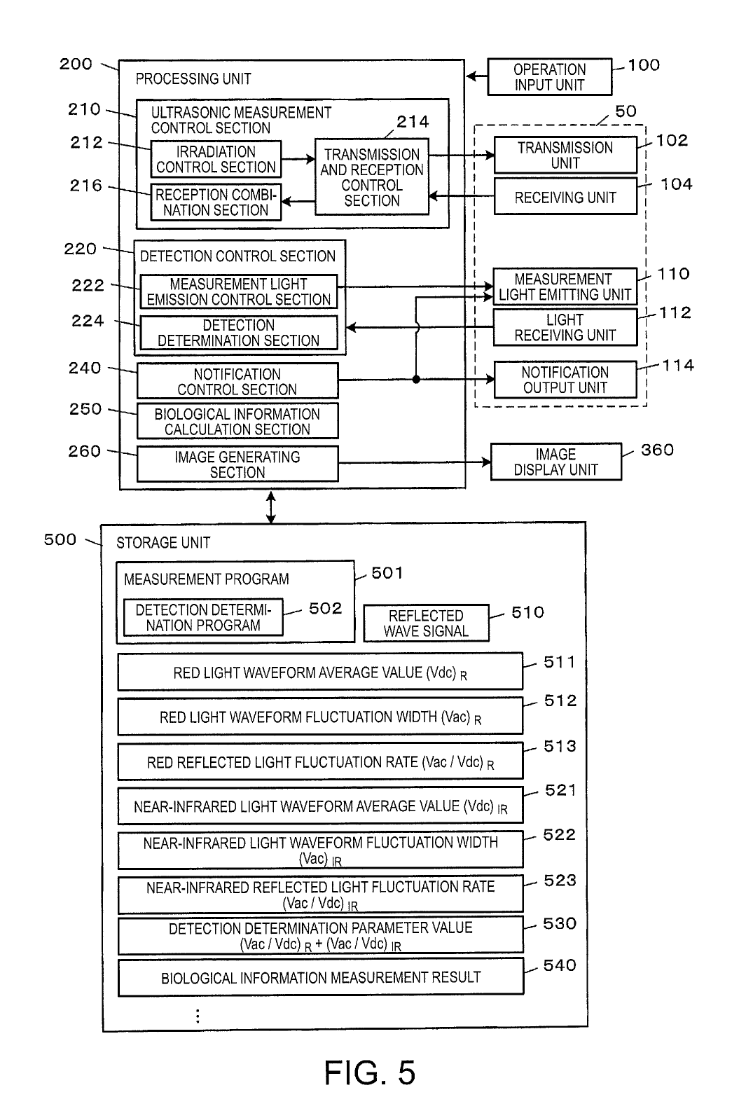

FIG. 5 is a block diagram showing an example of the functional configuration of the ultrasonic measuring device 10 of the present embodiment. The ultrasonic measuring device 10 includes an operation input unit 100, a transmission unit 102, a receiving unit 104, a measurement light emitting unit 110, a light receiving unit 112, a notification output unit 114, a processing unit 200, an image display unit 360, and a storage unit 500.

The operation input unit 100 receives various kinds of operation input by the operator, and outputs an operation input signal corresponding to the operation input to the processing unit 200. The operation input unit 100 can be realized by a button switch, a lever switch, a dial switch, a track pad, a mouse, or the like. The touch panel 12 and the keyboard 14 shown in FIG. 1 correspond to the operation input unit 100.

The transmission unit 102 emits an ultrasonic wave based on a pulse voltage.

The receiving unit 104 receives a reflected wave signal obtained when an ultrasonic wave irradiated from the transmission unit 102 is reflected in the body 4, converts the received wave signal into an electrical signal, and outputs the electrical signal. The ultrasonic element section 53 shown in FIGS. 2B and 2C corresponds to the transmission unit 102 and the receiving unit 104.

The measurement light emitting unit 110 emits measurement light for detecting the presence of tissue as an ultrasonic measurement target (measurement target tissue) so as to overlap the ultrasonic irradiation range of the transmission unit 102, that is, the measurement range of the ultrasonic measurement. The measurement light emitting unit 110 is realized by a light emitting element, an optical element, an optical filter, or the like. The first and second light emitting sections 54 and 56 shown in FIGS. 2A to 2C correspond to the measurement light emitting unit 110.

The light receiving unit 112 receives reflected light of the measurement light, converts the received reflected light into an electrical signal, and outputs the electrical signal. The light receiving unit 112 is realized by a known optical sensor, a known optical element, a known optical filter, or the like. The light receiving section 57 shown in FIGS. 2B and 2C corresponds to the light receiving unit 112.

The notification output unit 114 sends an output for notification of various situations in progress (measurement status) according to the measurement. For example, the notification output unit 114 can be realized by an image display device such as a liquid crystal panel display, an LED, a speaker, a vibrator, or the like. In the present embodiment, the touch panel 12 shown in FIG. 1 or the notification section 58 shown in FIGS. 2A and 2B corresponds to the notification output unit 114.

The processing unit 200 is realized by a microprocessor such as a CPU or a GPU, an ASIC, and an electronic component such as an IC memory, for example. The processing unit 200 performs control to receive data from each functional unit and to output data to each functional unit, determines a blood vessel position by performing various kinds of arithmetic processing based on a predetermined program or various kinds of data, and calculates biological information of the body 4. The processing unit 30 and the control board 31 shown in FIG. 1 correspond to the processing unit 200.

In the present embodiment, the processing unit 200 includes an ultrasonic measurement control section 210, a detection control section 220, a notification control section 240, a biological information calculation section 250, and an image generating section 260.

The ultrasonic measurement control section 210 includes an irradiation control section 212, a transmission and reception control section 214, and a reception combination section 216, and performs overall control of ultrasonic measurement. The sampling rate in the present embodiment is 20 times/second or more. As an example, measurement is assumed to be performed at 20 fps.

The irradiation control section 212 controls the timing of an ultrasonic pulse transmitted from the ultrasonic probe 50, and outputs the transmission control signal to the transmission and reception control section 214.

The transmission and reception control section 214 generates a pulse voltage according to the transmission control signal from the irradiation control section 212, and outputs the pulse voltage to the transmission unit 102. In this case, the timing of the output of the pulse voltage to each ultrasonic transducer can be adjusted by performing transmission delay processing. In addition, the transmission and reception control section 214 can perform the amplification or filtering processing of the reflected wave signal output from the receiving unit 104, and output the result to the reception combination section 216.

The reception combination section 216 generates a reflected wave signal by performing delay processing or the like when necessary to perform processing or the like according to so-called focusing of the received signal.

The detection control section 220 performs control related to the detection of tissue, which is an ultrasonic measurement target, by optical measurement. In the present embodiment, the detection control section 220 includes a measurement light emission control section 222 that controls the emission of the measurement light emitting unit 110 and a detection determination section 224 that receives an output signal from the light receiving unit 112, calculates various parameter values according to optical measurement, and detects measurement target tissue.

Various parameter values related to optical measurement for detecting the presence of measurement target tissue are stored in the storage unit 500. For example, a red light waveform average value 511, a red light waveform fluctuation width 512, a red reflected light fluctuation rate 513, a near-infrared light waveform average value 521, a near-infrared light waveform fluctuation width 522, a near-infrared reflected light fluctuation rate 523, and a detection determination parameter value 530 are calculated and stored.

The notification control section 240 controls the output of the notification output unit 114. In the present embodiment, when the presence of measurement target tissue has been detected by the detection control section 220, control to make the measurement light emitting unit 110 or the notification output unit 114 emit light in a predetermined emission pattern can be performed.

The biological information calculation section 250 calculates biological information regarding the measurement target tissue based on the reflected wave signal generated by the reception combination section 216. Examples of the biological information to be measured include vessel diameter, an arteriosclerosis index value, an elasticity index value, blood pressure, blood vessel age, and intima media thickness (IMT). The calculation result is stored in the storage unit 500 as a biological information measurement result 540. In addition, data of the reflected wave signal that is the basis of the calculation result can also be appropriately included in the biological information measurement result 540.

The image generating section 260 generates various operation images for screens, an image relevant to the detection of the presence of measurement target tissue, an image for displaying the measurement result of ultrasonic measurement and biological information measurement, an image for notification of the measurement status, and the like, and outputs them to the image display unit 360.

The image display unit 360 displays image data input from the image generating section 260. The touch panel 12 shown in FIG. 1 corresponds to the image display unit 360.

The storage unit 500 is realized by a storage medium, such as an IC memory, a hard disk, or an optical disk, and stores various programs or various kinds of data, such as data of the operation process of the processing unit 200. In FIG. 1, the storage medium 33 mounted on the control board 31 of the processing unit 30 corresponds to the storage unit 500. In addition, a connection between the processing unit 200 and the storage unit 500 is not limited to the connection using an internal bus circuit in a device, and may be realized by using a communication line, such as a local area network (LAN) or the Internet. In this case, the storage unit 500 may be realized by an external storage device that is different from the ultrasonic measuring device 10.

The storage unit 500 stores a measurement program 501, a reflected wave signal 510, the red light waveform average value 511, the red light waveform fluctuation width 512, the red reflected light fluctuation rate 513, the near-infrared light waveform average value 521, the near-infrared light waveform fluctuation width 522, the near-infrared reflected light fluctuation rate 523, the detection determination parameter value 530, and the biological information measurement result 540.

The processing unit 200 realizes the functions of the ultrasonic measurement control section 210, the detection control section 220, the notification control section 240, the biological information calculation section 250, the image generating section 260, and the like by reading and executing the measurement program 501.

In addition, when these functional sections are realized by hardware, such as an electronic circuit, a part of the program for realizing the functions can be omitted. For example, if the detection control section 220 is realized by the LSI or the like, a program portion for realizing the function of the detection control section 220, that is, a detection determination program 502 can be omitted.

The reflected wave signal 510 is data of the reflected wave signal acquired by ultrasonic measurement, and is generated for each frame by the ultrasonic measurement control section 210. For example, in one reflected wave signal 510, identification information (Tr) of the ultrasonic transducer and measured frame identification information (fr) are stored so as to match each other.

In addition to these, the storage unit 500 can appropriately store data required for the determination of blood vessel positions and the calculation of biological information, such as various flags and counter values for checking time.

### Explanation of the Process Flow

Next, the operation of the ultrasonic measuring device 10 will be described.

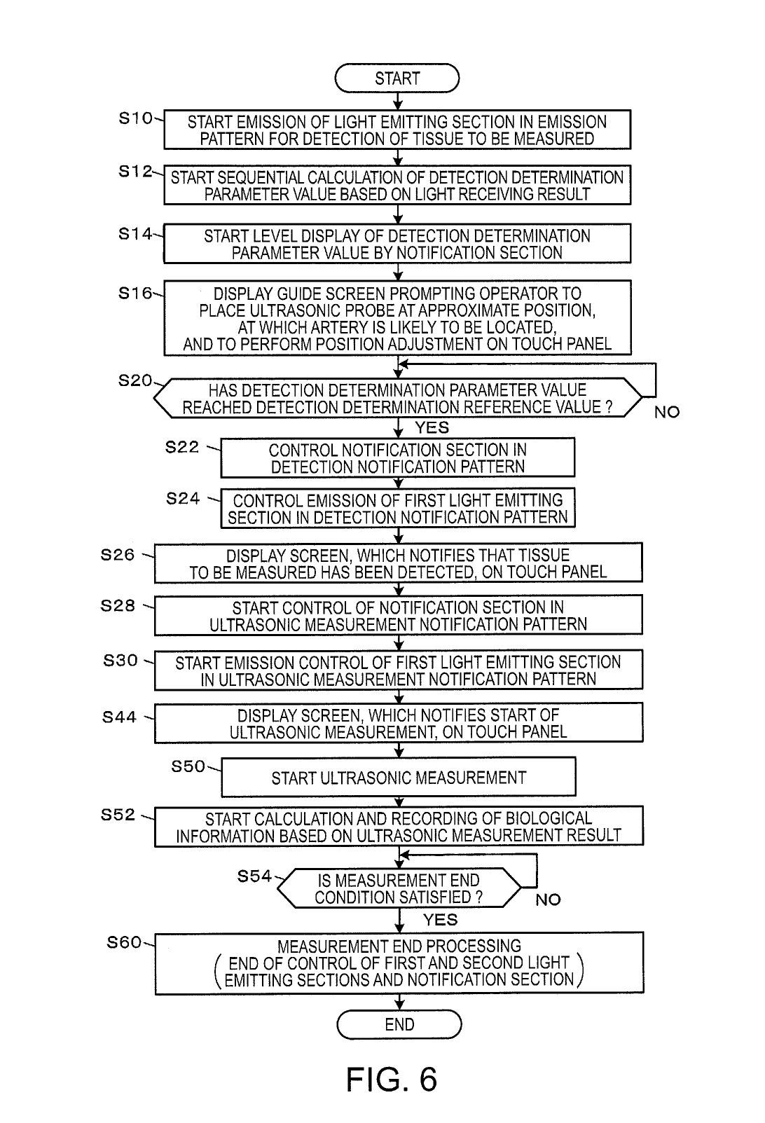

FIG. 6 is a flowchart for explaining the flow of the process according to the detection of the presence of measurement target tissue and the ultrasonic measurement of the ultrasonic measuring device 10.

First, the processing unit 200 starts the emission of the first and second light emitting sections 54 and 56 in an emission pattern of the detection amount of measurement target tissue (for example, always-lighting state) (step S10). Then, sequential calculation of the detection determination parameter value 530 is started based on the light receiving result of the light receiving section 57 (step S12), and the level display of the detection determination parameter value 530 by the notification section 58 is started (step S14). In addition, since preparation for detection of the presence of measurement target tissue has been completed, a guide image prompting the operator to place the ultrasonic probe 50 at an approximate skin surface position, at which the measurement target tissue (in the present embodiment, the blood vessel 6, more specifically, an artery) is likely to be located under the skin, and to perform position adjustment is generated, and the guide image is displayed on the touch panel 12 (step S16).

When the detection determination parameter value 530 which is sequentially calculated reaches a predetermined detection determination threshold value (YES in step S20), the processing unit 200 controls the notification section 58 in a detection notification pattern (step S22), and controls the emission of the first light emitting section 54 in a detection notification pattern (step S24). Then, a screen notifying that the measurement target tissue has been detected is displayed on the touch panel 12 (step S26).

Then, the processing unit 200 starts a control based on the ultrasonic measurement pattern for the notification section 58 (step S28), and starts a control to make the first light emitting section 54 emit light according to the ultrasonic measurement pattern (step S30). Then, a screen notifying that the ultrasonic measurement will start is displayed on the touch panel 12 (step S44).

Then, the processing unit 200 starts the ultrasonic measurement (step S50). Calculation and recording of biological information based on the ultrasonic measurement results are started (step S52). In addition, it is preferable to perform countdown processing before the start. In addition, light emission from the first and second light emitting sections 54 and 56 may also be appropriately stopped.

After starting the ultrasonic measurement, when it is detected that a predetermined end condition is satisfied (YES in step S54), the processing unit 200 performs measurement end processing (step S60) to end a series of processes.

As described above, according to the present embodiment, it is possible to realize an auxiliary function of detecting the position of an ultrasonic measurement target. In addition, in order to realize an auxiliary function, an ultrasonic array of a two-dimensional array type as in the related art is not required, and only the first and second light emitting sections 54 and 56 are prepared. Therefore, it is possible to realize an auxiliary function at a lower cost. Also regarding the notification of the measurement status, if light leakage from the structural section 59 is sufficient, the notification section 58 can also be omitted. In this case, it is possible to realize an auxiliary function at a lower cost.

In the related art, in particular, in the technique disclosed in JP-A-2002-11008, the operator should observe an ultrasonic image displayed on the monitoring screen, which is different from using the hand to manipulate the ultrasonic probe, and read and interpret the presence of measurement target tissue from the ultrasonic image. For this reason, skill or concentration has been required in order to adjust the position of the ultrasonic probe. In the present embodiment, however, when measurement target tissue is detected, a notification that the measurement target tissue has been detected by the ultrasonic probe is given. Therefore, the operator has only to focus on the hand to manipulate the ultrasonic probe and does not need to read and interpret the ultrasonic image, and concentration therefor is not required. As a result, it is possible to significantly reduce the operation load of the operator.

In the present embodiment, measurement light beams of different wavelengths are emitted from the first and second light emitting sections 54 and 56. However, either of the first and second light emitting sections 54 and 56 can also be omitted.

In parallel to ultrasonic measurement or apart from the ultrasonic measurement, the first and second light emitting sections 54 and 56 and the light receiving section 57 can be made to function as a known reflection type pulse oximeter.

### Second Embodiment

Next, a second embodiment to which the invention is applied will be described.

The present embodiment is realized in basically the same manner as the first embodiment, but is different from the first embodiment in that not the processing unit 30 but the ultrasonic probe performs control regarding optical measurement for detecting the presence of measurement target tissue. Hereinafter, differences from the first embodiment will mainly be described, and the same components as in the first embodiment are denoted by the same reference numerals and repeated explanation thereof will be omitted.

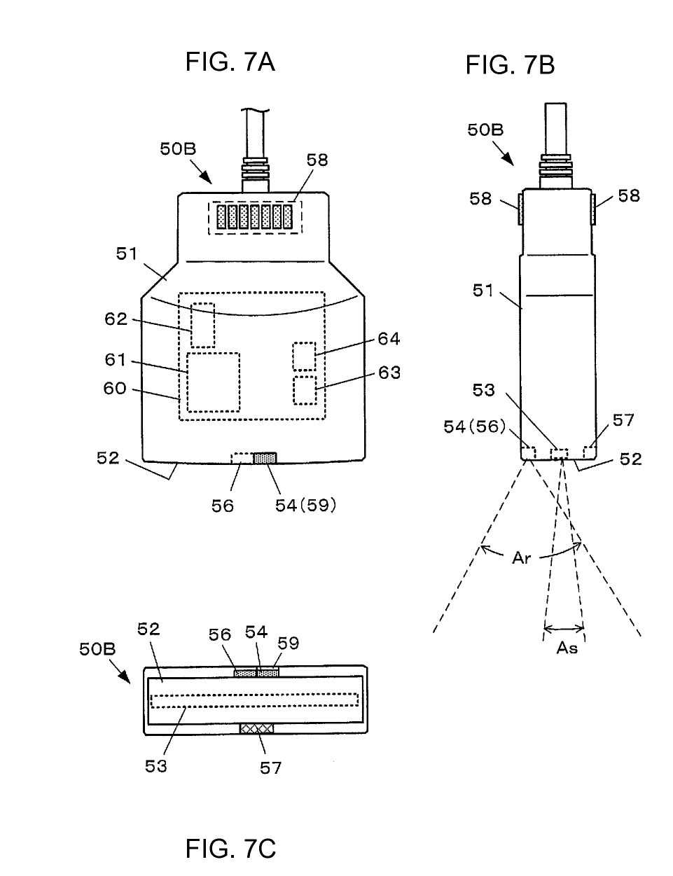

FIGS. 7A to 7C are diagrams of three sides showing an example of the configuration of an ultrasonic probe 50B in the present embodiment. The ultrasonic probe 50B of the present embodiment includes a probe control board 60 in the main body case 51. A CPU 61, an IC memory 62, an interface IC 63 for input and output of a signal to control a first light emitting section 54 or a second light emitting section 56 and a notification section 58, and a communication IC 64 for data communication with a processing unit 30 are mounted on the board.

The CPU 61 reads a program stored in the IC memory to execute various kinds of arithmetic processing for controlling the first light emitting section 54 or the second light emitting section 56 and the notification section 58.

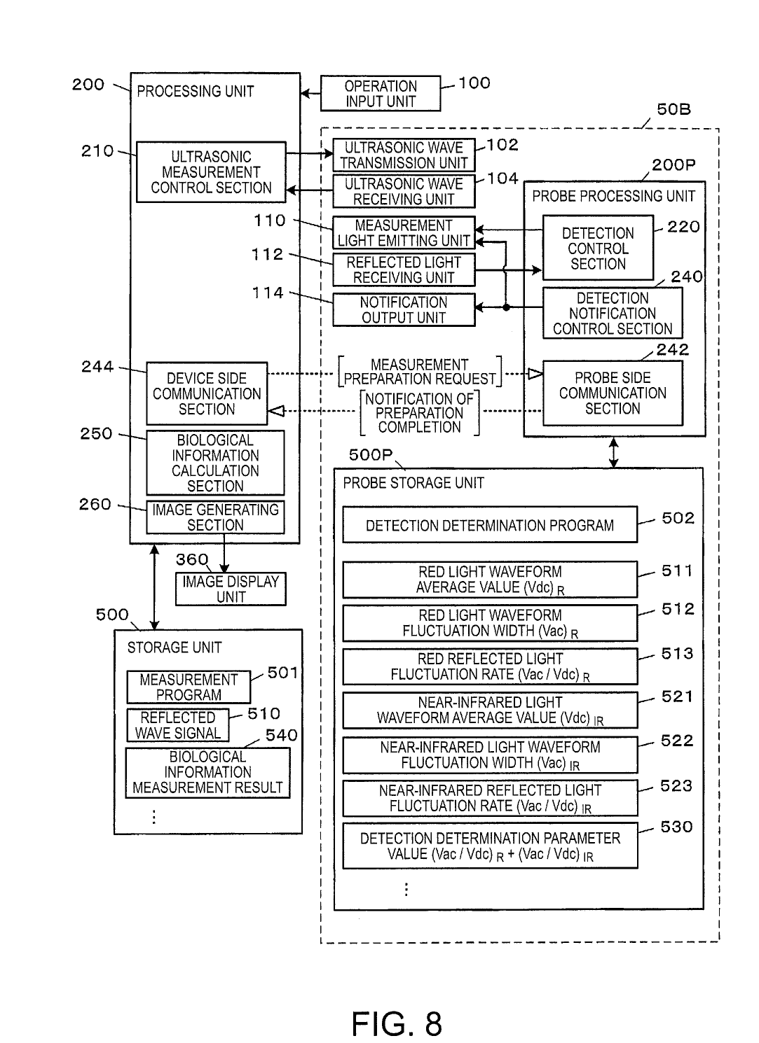

FIG. 8 is a functional block diagram illustrating the functional configuration in the present embodiment.

In the present embodiment, the detection control section 220 and the notification control section 240 are included not in the processing unit 200 but in a probe processing unit 200P (corresponding to the probe control board 60 in FIG. 7A) of the ultrasonic probe 50B, in contrast with the first embodiment.

In addition, the probe processing unit 200P includes a probe side communication section 242 (corresponding to the communication IC 64 in FIG. 7A), and performs data communication with a main device side communication section 244 of the processing unit 200.

The probe processing unit 200P realizes a function as the detection control section 220 and the notification control section 240 by reading and executing a detection determination program 502 stored in a probe storage unit 500P (corresponding to the IC memory 62 in FIG. 7A). This does not apply when the detection control section 220 and the notification control section 240 are realized by hardware, such as an IC chip.

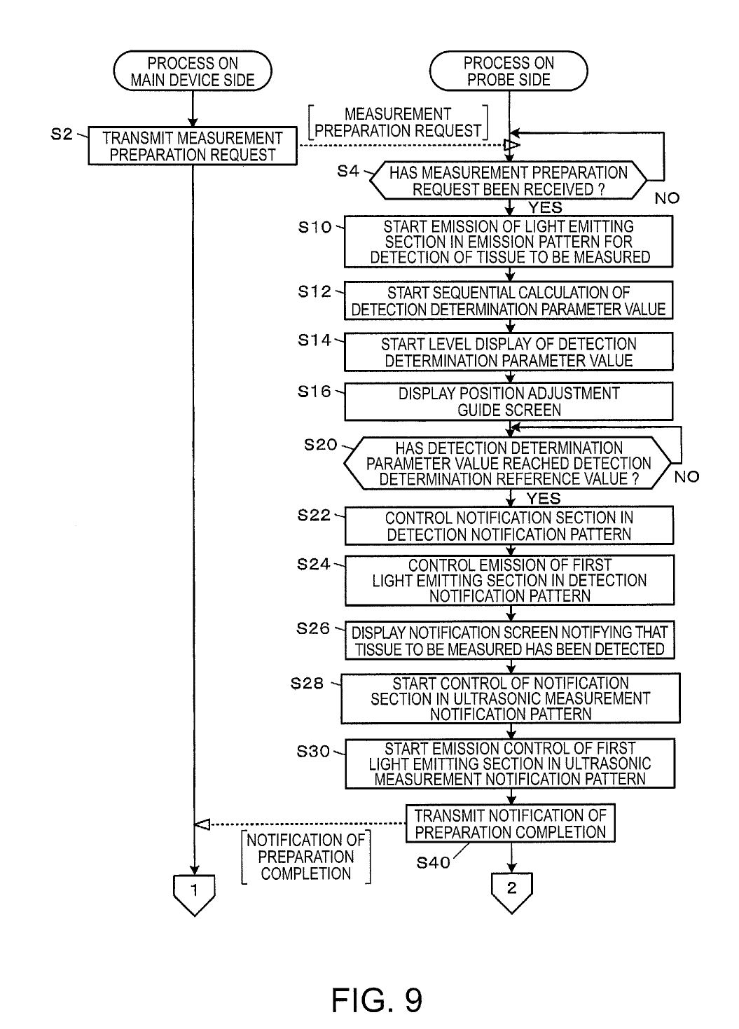

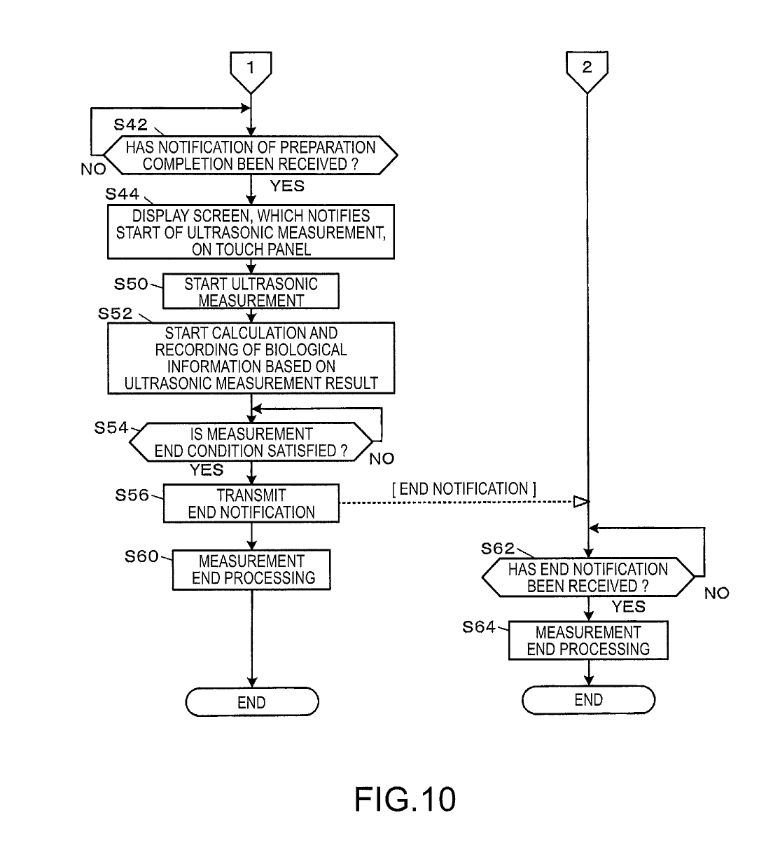

FIGS. 9 and 10 are flowcharts for explaining the flow of the process according to the detection of the presence of measurement target tissue and the ultrasonic measurement of the ultrasonic measuring device 10 in the present embodiment.

The process flow of the present embodiment is basically the same as that of the first embodiment, but the following points are different. That is, when the processing unit 200 transmits a measurement preparation request to the probe processing unit 200P (step S2), the probe processing unit 200P receives the request (YES in step S4), executes steps S10 to S30, and transmits the notification of preparation completion to the processing unit 200 (step S40).

Referring to the flowchart shown in FIG. 10, when the notification of preparation completion is received (YES in step S42), the processing unit 200 executes steps S44 to S54. Then, when the end condition is satisfied (YES in step S54), the notification of measurement completion is transmitted to the probe processing unit 200P (step S56), and measurement end processing is performed (step S60).

On the other hand, when the end notification is received (YES in step S62), the probe processing unit 200P performs measurement end processing, such as stopping the emission of the first light emitting section 54 or the second light emitting section 56 and ending the notification of the notification section 58 (step S64).

### MODIFICATION EXAMPLES

While the embodiments to which the invention is applied have been described above, additions, omissions, and modifications of constituent components can be appropriately made without being limited to the embodiments described above.

For example, although the notification section 58 is separately provided in the embodiment described above, the notification section 58 may be omitted, and the notification function of the measurement status may be realized by the control of the emission pattern of the first light emitting section 54 and the structural section 59.

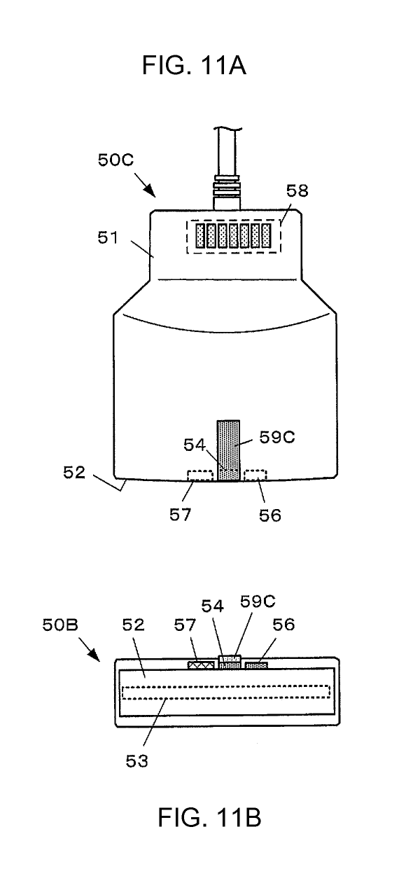

For example, as an ultrasonic probe 50C shown in FIG. 11A, a configuration can also be adopted in which the first and second light emitting sections 54 and 56 and the light receiving section 57 are provided so as to be closer to one side of the array of the ultrasonic element section 53. In addition, instead of the structural section 59 in the first or second embodiment, a light guide member 59C through which light from the first light emitting section 54 is guided so as to be appropriately diffused may be provided.

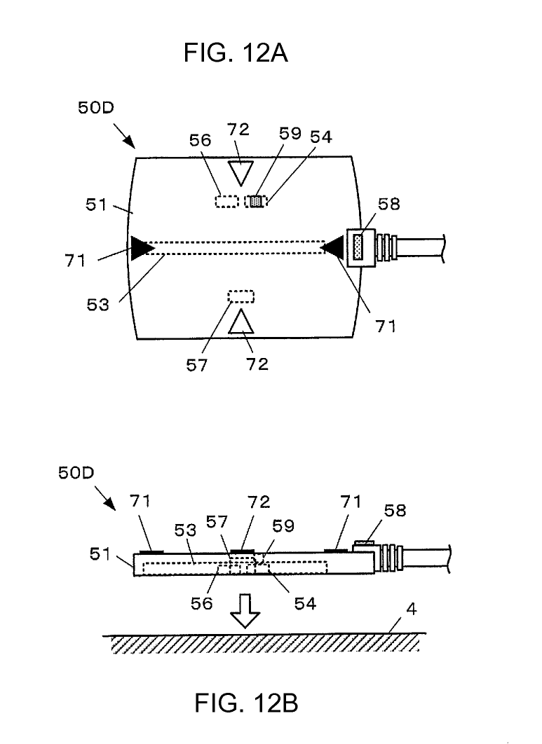

In addition, the shapes of the ultrasonic probes 50 to 50C are not limited to the stick shape. For example, as an ultrasonic probe 50D shown in FIGS. 12A and 12B, it is possible to use a plate-shaped or sheet-shaped ultrasonic probe that can be attached to the skin surface of the body 4 with a gel or the like. In this configuration, an arrangement direction marker 71 showing an array of the ultrasonic element section 53 and an optical measurement direction marker 72 showing a direction in which the first and second light emitting sections 54 and 56 are connected to the light receiving section 57 are preferably provided on the top surface (surface facing the operator) of the main body case 51. The arrangement direction marker 71 or the optical measurement direction marker 72 may be realized by an LED or the like so as to function as the notification section 58.

The entire disclosure of Japanese Patent Application No. 2013-229058, filed on Nov. 5, 2013 is expressly incorporated by reference herein.

What is claimed is:

1. An ultrasonic probe, comprising:
an ultrasonic element section that performs ultrasonic measurement of measurement target tissue in a body; and
a light emitting section and a light receiving section that emit and receive measurement light for detecting the measurement target tissue by optical measurement and that are provided such that a propagation range of the measurement light in the body overlaps a measurement range of the ultrasonic element section.

2. The ultrasonic probe according to claim 1,
wherein the ultrasonic element section includes an ultrasonic element array, and
the ultrasonic element array is disposed between the light emitting section and the light receiving section.

3. The ultrasonic probe according to claim 1, further comprising:
a notification section that notifies that the measurement target tissue has been detected by the optical measurement.

4. The ultrasonic probe according to claim 3,
wherein the notification section is a structural section through which light from the light emitting section leaks or is guided to a side of the ultrasonic probe, and
the notification is given by controlling an emission pattern of the light emitting section.

5. The ultrasonic probe according to claim 1,
wherein the measurement target tissue is a blood vessel.

6. An ultrasonic measuring device, comprising:
the ultrasonic probe according to claim 1; and
a detection control section that detects the measurement target tissue by controlling the light emitting section and the light receiving section to perform the optical measurement.

7. An ultrasonic measuring device, comprising:
the ultrasonic probe according to claim 2; and
a detection control section that detects the measurement target tissue by controlling the light emitting section and the light receiving section to perform the optical measurement.

8. An ultrasonic measuring device, comprising:
the ultrasonic probe according to claim 3; and
a detection control section that detects the measurement target tissue by controlling the light emitting section and the light receiving section to perform the optical measurement.

9. An ultrasonic measuring device, comprising:
the ultrasonic probe according to claim 4; and
a detection control section that detects the measurement target tissue by controlling the light emitting section and the light receiving section to perform the optical measurement.

10. An ultrasonic measuring device, comprising:
the ultrasonic probe according to claim 5; and
a detection control section that detects the measurement target tissue by controlling the light emitting section and the light receiving section to perform the optical measurement.

11. An ultrasonic measuring device, comprising:
the ultrasonic probe according to claim 4;
a detection control section that detects the measurement target tissue by controlling the light emitting section and the light receiving section to perform the optical measurement; and
a notification control section that makes the light emitting section emit light in a predetermined emission pattern when the measurement target tissue is detected by the detection control section.

12. An ultrasonic measuring device, comprising:
the ultrasonic probe according to claim 3 in which the notification section is a display section;
a detection control section that detects the measurement target tissue by controlling the light emitting section and the light receiving section to perform the optical measurement and calculating an index value indicating a level to detect the measurement target tissue; and
a notification control section that performs display control of the notification section according to the index value calculated by the detection control section.
